(19) United States
(12) Patent Application Publication
(10) Pub. No.: US 2009/0053304 A1
(43) Pub. Date: Feb. 26, 2009

(54) Composition and method of producing a taste masking formulation of laxatives for bowel cleaning preparation prior to colonoscopy

(76) Inventors: Jinling Chen, Houston, TX (US); Lawrence Thomas Chang, Houston, TX (US)

Correspondence Address: Jinling Chen, 4443 Sterling Wood Way, Houston, TX, 77059, US

(21) Appl. No.: 12/220,296

(22) Filed: Jul. 23, 2008

Related U.S. Application Data
(60) Provisional application No. 60/957,513, filed on Aug. 23, 2007.

(51) Int. Cl. A61K 31/78; A61K 9/48

(52) U.S. Cl. 424/451; 424/78.01

(57) ABSTRACT

This invention relates to a solid taste masking formulation of laxatives which can be dispersed in water for oral use for bowel cleaning preparation prior to colonoscopy procedures. This invention also relates to the methods to produce the taste masking laxative formulations.

### RELATED APPLICATION

The present application claims the priority U.S. provisional patent application 60/957,513, filed on Aug. 23, 2007.

### FIELD OF THE INVENTION

This invention relates to a composition and a method of producing a taste masking formulation of laxatives for bowel cleaning preparation prior to a colonoscopy procedure. In particular, this invention relates to a solid taste masking formulation that can be dispersed in liquid such as water or taken with the liquid orally for the purpose of bowel cleaning before a colonoscopy procedure and a method to produce such a formulation.

### BACKGROUND OF THE INVENTION

Nearly 150,000 people are informed that they have colorectal cancer (cancers of the colon and the rectum) each year. 55,000 people die every year. Colorectal cancer is the third most common cancer in both men and women. It can be effectively treated when detected early. Therefore, early detection of colorectal cancer is of utmost importance.

One of the best tools for detecting colon cancer is a colonoscopy. The American Cancer Society (ACS) recommends regular colonoscopy screening beginning at age 50. In addition to cancer screening and detection, a colonoscopy is used in the diagnosis of inflammatory bowel diseases (Ulcerative Colitis and Crohn's Disease), hemorrhoids, diverticulosis and other illnesses related to the colon.

Despite the efforts of many, colonoscopy screening rates among those who could benefit the most remain low. Studies set out to determine exactly which elements of colonoscopy deterred patients from getting the procedure show consistently that the bowel preparation ranked number one as a deterrent. Compared to the colonoscopy procedure itself, which is generally well tolerated, the bowel preparation procedure prior to testing is often described as highly uncomfortable, which discourages many people from taking the routine screening. Before the colonoscopy procedure, the bowel must be thoroughly cleansed so that the intestinal walls can be seen clearly by the doctor in order for the colonoscopy to be successful. To prepare for the test, one needs to limit or eliminate solid foods for 1-2 days, then take laxatives orally to clean the bowel.

Commercially-available bowel preparation products typically contain large amounts of polyethylene glycol and electrolytes [1-3]. For example, one of the current leading prescription laxatives for colonoscopy preparation, NuLYTELY®, is an oral solution of polyethylene glycol 3350 and electrolytes (sodium chloride, sodium bicarbonate, and potassium chloride). A large amount (4 liters) of this salty and foul-tasting solution must be taken orally to clean up the bowel. Most people describe it as very unpleasant and many are unwilling or unable to take it.

Some of the recently patented bowel cleaning formulations also contain compositions that are difficult to take by patients. For example, U.S. Pat. No. 7,169,381 describes a formulation that contains 0 to 350 g polyethylene glycol, 3 to 20 g of an ascorbic acid, a salt of ascorbic acid, a sulfate salt, and optionally one or more electrolytes selected from sodium chloride, potassium chloride, and sodium hydrogen carbonate, and preferably also appropriate flavoring.

Efforts have been made to reduce the intolerability of the bowel cleaning experience by improvement of formulation. U.S. Pat. No. 6,946,149 describes a low volume, hyper-osmotic solution consisting of sulfate salts with and without polyethylene glycol. Flavoring is often added to the formulation. Nevertheless, the bowel cleaning process remains the major reason for people not wanting to take a colonoscopy.

Therefore, a palatable oral formulation for the bowel preparation prior to a colonoscopy procedure is highly desirable. Such a formulation can improve tolerability of the overall colonoscopy procedure. The positive experience by patients using the palatable bowel preparation formulation would encourage more patients to have the procedure done and significantly increase colonoscopy screening rates among those who could benefit the most.

Oral taste masking formulations are known. These formulations are mostly often used to mask bitter-tasting pharmaceutically active ingredients. In comparison, this invention relates to a taste masking formulation of laxatives where there is no pharmaceutically active ingredient. Unlike most of the active pharmaceutical ingredients which need to be absorbed into the body to be biologically effective, the ingredients in the laxative formulation are eliminated from the body quickly without absorption while still serving the bowel cleaning purpose.

Apart from these differences, there are additional challenges involved in making a taste masking formulation of laxatives. For example, (1), all the excipients used for taste masking purposes, which include the primary taste masking excipient and any other co-processing excipients such as plasticizers, dispersants, etc., need to be fully soluble in the gastric fluid to avoid retainment in the gastrointestinal track. Any un-dissolved solid particles would negatively affect the colonoscopy imaging; (2), unlike most pharmaceutical tablets or particles, many commonly used laxative formulations contain large amounts of low-melting materials such as PEG3350, which begins to melt at approximately 50° C. The formulation manufacturing process should preferably be operated at a lower temperature to avoid melting of the PGE3350 during the process.

### DETAILED DESCRIPTION OF THE INVENTION

This invention provides a palatable oral bowel preparation formulation and a method to prepare such a formulation. In particular, this invention provides a taste masking solid formulation of laxative which can be dispersed in a liquid such as water or taken with the liquid for oral use. Once the solids reach the stomach, the solid laxative particles become fully water soluble in the gastric fluid to be available for bowel cleaning purposes.

According to one aspect of the invention, a taste masking formulation with a pH-dependant coating material is provided for the purpose of taste masking. The polymer coating material that is provided is water insoluble at neutral pH conditions and therefore stays intact in the mouth to mask the taste of the laxative ingredients. The same polymer material is water soluble at acidic pH conditions and therefore becomes soluble in the acidic gastric fluid to release the water soluble laxative ingredients for bowel cleaning.

In one embodiment, the coating contains one or more types of pH-dependent materials which are water insoluble at neutral pH and basic conditions but water soluble at acidic pH conditions so that it stays intact in the mouth but is dissolved in the acidic gastric fluid to release the laxative ingredients for bowel cleaning.

Preferably, the pH-dependent excipients should have relatively low water permeability so that they protect water from penetrating through the coating layer into the core of the particles to dissolve the unfriendly-tasting laxative ingredients.

In one embodiment, the coating contains at least one pH-dependant material with or without other pharmaceutically acceptable excipients.

In another embodiment, the coating contains one or more pH-dependant material and other pharmaceutically acceptable excipients that are fully soluble or dispersible in the acidic gastric fluid.

In another embodiment of the invention, a taste masking formulation with a coating material that has a temperature dependent solubility is provided for the purpose of taste masking. The coating material that is provided is insoluble in aqueous medium at or below the ambient temperature and therefore stays intact in the drinkable liquid such as water to mask the taste of the laxative ingredients. The same coating material is soluble in the warm gastric fluid to release the water soluble laxative ingredients for bowel cleaning once it reaches the stomach.

In another embodiment of the invention, a method is provided to produce the taste masking laxative formulation. The ingredients used for bowel cleaning purposes are first made into particles or beads, followed by surface coating or encapsulation with a layer of taste masking materials for oral use.

In one embodiment of the invention, the particles or beads can be coated by any type of coating or encapsulation process.

In one embodiment, the particles or beads are coated using a spray coating process, such as fluid bed coating or pan coating.

In one embodiment, the particles or beads can be encapsulated using any type of encapsulation process such as surface deposition, interfacial polymerization, or physical coating.

According to another aspect of the invention, the coating or encapsulation process can be operated at any suitable processing temperature. Preferably, the coating or encapsulation is processed at a temperature equal to or below the melting temperature of the laxative ingredients in the core to avoid melting of these materials during the process.

According to another aspect of the invention, the uncoated particles or beads can be of any geometric shape, color, and size.

In one embodiment of the invention, the laxative ingredients are mixed together and processed to form uniform particles before coating or encapsulation.

In another embodiment, the laxative ingredients are process into layered or other types of non-uniform mixtures before coating.

In another embodiment, the laxative ingredients are processed into tablets before coating.

In yet another embodiment, the laxative ingredients are coated or encapsulated separately.

In one embodiment, the invention provides a taste masking laxative formulation in capsule form.

In another embodiment of the invention, a method is provided to produce a taste masking laxative capsule formulation.

In yet another embodiment, this invention provides a taste masking formulation with taste masking excipients incorporated in the laxative ingredient particles.

### DETAILED DESCRIPTION OF THE INVENTION

### I. A TASTE MASKING ORAL SOLID LAXATIVE FORMULATION FOR BOWEL CLEANING PURPOSES

This invention provides a palatable taste masking oral bowel preparation formulation. The taste masking solid formulation of laxative can be dispersed in a drinkable liquid such as water or it can be taken with the liquid orally. The ingredients used for bowel cleaning purposes are surface coated or encapsulated with a layer of pH-dependent material for taste masking purposes. The coating material is water insoluble at neutral or basic pH conditions and therefore stays intact in mouth to mask the taste of the laxative ingredients. The same coating material is water soluble at low acidic pH conditions and therefore becomes solubilized in the acidic gastric fluid once it reaches the stomach to release the laxative ingredients, which are fully water soluble, for bowel cleaning purposes.

The materials that have pH-dependent solubility profiles are normally pharmaceutically acceptable excipients which contain one or more cationic functional groups such as primary or secondary amines. The amine group remains in neutral form at pH above its ionization constant (pKa), but becomes predominantly positively charged by protonation at pH below its pKa, which significantly increases its aqueous solubility. The pKa value of an amine group varies with the chemical structure of the molecules. Preferable materials have pKa values of 2-6. The pKa values of the coating material can be estimated by calculation or determined by potentiometric, spectrophotometric, or other industrial standard methods.

Preferably, the pH-dependent ingredient should have relatively low water permeability at neutral pH conditions so that it prevents water from penetrating through the coating layer into the particle core to dissolve the unfriendly-tasting laxative ingredients.

Alternatively, the ingredients used for bowel cleaning purposes are surface coated or encapsulated with a layer of coating material that has a temperature dependent solubility for the purpose of taste masking. The coating material that is provided is insoluble in aqueous medium at or below room temperature and therefore stays intact in the drinkable liquid such as water to mask the taste of the laxative ingredients. The same coating material is soluble in the warm gastric fluid to release the water soluble laxative ingredients for bowel cleaning once it reaches the stomach.

According to the invention, one or more other excipients can be used alone with the pH-dependent or the temperature-dependent excipient in the coating layer. Those materials serve the purposes of plasticizer, dispersant, glidant, etc. to facilitate the coating process, or to modify the surface properties of the coating.

For example, a plasticizer may be used to reduce the processing temperature during the coating to avoid the laxative ingredients from melting in the core during the coating process or to reduce the heat exposure of the laxative ingredients for stability purposes. In addition, a plasticizer may reduce the rigidity of the coating material, making it stronger and more flexible.

A dispersant may be used to reduce the particle size of the pH-dependent or temperature-dependent materials in the coating suspension to ensure a uniform and smooth coating coverage.

A glidant may be used to improve the flow property or other physical properties of the coated laxative particles.

Preferably, all excipients used for coating are soluble or highly dispersible in warm acidic aqueous media to avoid retainment in the gastrointestinal track.

In one embodiment of the invention, the coating contains one or more pH-dependant or temperature-dependent materials and other pharmaceutically acceptable excipients that are fully soluble or dispersible in the acidic gastric fluid.

According to the invention, the surface taste masking coating layer preferably comprises from about 0.1% to about 95%, more preferably from about 0.5% to about 50%, and most preferably from about 1% to about 10%, by weight, of the whole formulation.

The coating layer, according to the invention, comprises of 5% to 100%, preferably 30% to 100%, and more preferably 70% to about 100% of the excipient with pH-dependent or temperature-dependent solubility within the coating, by weight, for taste masking purposes.

According to another aspect of the invention, the uncoated particles or beads are of any geometric shape, e.g., beads, tablets, etc. The uncoated particles can also be of any color and size.

The laxative ingredients can be mixed together and processed to form uniform particles before coating or encapsulation. Alternatively, the laxative ingredients can be processed into layered or other types of non-uniform mixtures before coating.

In yet another embodiment, the laxative ingredients can be coated or encapsulated separately.

### II. A METHOD TO PRODUCE TASTE MASKING ORAL SOLID LAXATIVE FORMULATION FOR BOWEL CLEANING PURPOSES

A method is provided to produce the taste masking laxative formulation. The ingredients used for bowel cleaning purposes are first made into particles or beads, followed by surface coating or encapsulation with a layer of taste masking materials for oral use.

According to the invention, the particles can be coated by any coating or encapsulation process.

In one embodiment, the particles are coated using a spray coating process. Examples of suitable coating processes include, but are not limited to: fluid bed coating, pan coating, surface deposition, and interfacial polymerization. The preferred coating process is the fluid bed coating process.

In one embodiment, the particles are encapsulated using an encapsulation process. Examples of suitable encapsulation processes include, but are not limited to: surface deposition and interfacial polymerization. The preferred encapsulation process is the surface deposition process.

A wide variety of solvents can be used to dissolve or disperse the taste masking excipients. Suitable solvents include, but are not limited to: water, ethanol, isopropyl ethanol, acetone, methylene dichloride, supercritical fluid carbon dioxide, and the mixture of one or more solvents.

The coating or encapsulation process can generally be carried out at a wide range of temperatures. An effective operating temperature range for an aqueous based process can be from about 0° C. to about 99° C., preferably from 5° C. to 50° C.

In practice, the coating or encapsulation is preferably processed at a temperature equal to or below the melting temperature of the laxative ingredients in the uncoated beads to avoid the melting of these materials during the process.

For example, some laxative formulations contain a large percentage of a low melting solid such as PEG3350, which has a melting temperature of about 50° C.-60° C. The melting of PEG during the coating or encapsulation process would complicate the operation. Therefore, in this case, the preferred operation temperature is below 50° C. More preferably, the process is close to or below room temperature to avoid possible chemical decompositions of the laxative ingredients in the core and the coating ingredients at the surface to enhance the formulation stability.

### III. A TASTE MASKING ORAL LAXATIVE CAPSULE FORMULATION FOR BOWEL CLEANING PURPOSES

In one embodiment of the invention, a taste masking formulation of the laxative in capsule forms is provided. In particular, this invention provides a taste masking formulation of the laxative in capsule form which can be dispersed in water or any other suitable liquid and taken orally.

Preferably, relatively small-sized capsules such as mini-capsules are used for easy swallowing.

The laxative ingredients in the capsules can be in solid or liquid forms. The laxatives in liquid form may be used to provide fast dissolution of the laxative ingredients from the capsules once it reaches the stomach.

The capsules are coated with a layer of pH-dependant or temperature-dependent materials for taste masking purposes.

The pH-dependant coating material that is provided is insoluble in aqueous medium at neutral pH and therefore stays intact in a drinkable liquid such as water and in the month to mask the taste of the laxative ingredients. The same polymer material is water soluble at low pH medium and, therefore, once the coated capsules reach the stomach, the coating dissolves in the gastric fluid, followed by the dissolution of capsule shell, to allow the laxative ingredients inside the capsule, which are fully water soluble, to be released and solubilized in the gastric fluid for bowel cleaning purposes.

Alternatively, the coating material that is provided is has a temperature dependent solubility profiles. The coating material that is provided is insoluble in aqueous medium at or below room temperature and therefore stays intact in the drinkable liquid such as water to mask the taste of the laxative ingredients. The same coating material is soluble in the warm gastric fluid to release the water soluble laxative ingredients for bowel cleaning once it reaches the stomach.

### IV. A METHOD TO PRODUCE TASTE MASKING CAPSULE FORMULATION OF LAXATIVES FOR BOWEL CLEANING PURPOSES

In another embodiment of the invention, a method is provided to produce the taste masking capsule formulation of the laxative. The ingredients used for bowel cleaning purposes are first filled into capsules. The capsules are then coated by a suitable coating process. Examples of suitable coating processes include, but are not limited to: spray coating processes such as fluid bed coating or pan coating.

The laxative ingredients can be mixed together and filled into capsules, or the ingredients can be filled into separate capsules before coating.

### V. EXAMPLES

### Example 1

A 20 gram sample of laxative ingredients containing polyethylene glycol 3350 (80% wt/wt), sodium chloride (10% wt/wt), and sodium phosphate (10% wt/wt) is mixed in a plastic bag for 5 minutes. The mixture is then heated to about 60-70° C. to melt the polyethylene glycol 3350. The liquefied mixture is stirred by a spatula to ensure the homogeneity of the sample. It was then allowed to cool slowly to solidify. While the sample is still soft, the sample is extruded and cut into beads of approximately 2 mm in diameter and 2 mm in length.

A formulation of the coating solution is prepared in a separate container. The composition of the coating suspension is given in the table below.

| Ingredient | Amount (grams) |
|---|---|
| Eudragit E PO | 10.00 |
| Sodium Lauryl Sulfate | 1.05 |
| Glyceryl Monostearate | 0.15 |
| Polyethylene Glycol 6000 | 2.00 |
| Water | 86.80 |

Wherein the Eudragit E PO is a poly (butyl methacrylate, (2-dimethyl aminoethyl methacrylate, methyl methacrylate) copolymer, supplied by Degussa.

To prepare the coating solution, sodium lauryl sulfate is first dissolved in water using a propeller. Eudragit E PO is slowly suspended in this mixture and the stirring continued for another 10 minutes. Then glyceryl monostearate and polyethylene glycol 6000 are added. The suspension is stirred for another 5 hours approximately until a colloidal solution is formed.

The beads are loaded into a miniature scale rotating coating pan. While the beads are rotated, the coating solution is sprayed slowly. Hot air is used to facilitate the water evaporation and drying process. The sample temperature is kept between 25-32° C. during the coating process.

Samples are taken periodically and weighed to determine the weight gain. After a total weight gain of 10% is achieved in about 2 hours, the spray is stopped and the coated beads are dried in the same fluid bed at 35° C. for 2 hours. The fluid bed is then turned off and the coated beads are collected for taste testing.

Taste Testing Sample 1: A 1 gram sample of the laxative ingredient mixture (PEG 3350, sodium, and sodium sulfate) is added to 10 mL of cold water in a cup. The sample is mixed until the laxative ingredients are fully dissolved. The solution tastes salty and bitter.

Taste Testing Sample 2: A 1 gram sample of the uncoated beads of the laxative ingredient mixture (PEG 3350, sodium, and sodium sulfate) is added to 10 mL of cold water in a cup. One minute after the addition of the beads to water, the solids are partially dissolved. The sample tastes salty and somewhat bitter. After 10 minutes, the beads are fully dissolved, and the samples taste bitter, similar to sample 1.

Taste Testing Sample 3: A 1.1 gram sample of the coated beads with 10% coating is added to 10 mL of cold water in a cup. One minute after the addition of the beads to water, the beads remain unchanged in water and the sample has no taste. After 10 minutes, the beads remain in the water and the samples still have no taste.

### Example 2

A sample of laxative ingredients containing PEG 3350 (80% wt/wt), sodium chloride (10% wt/wt), and sodium phosphate (10% wt/wt) is mixed in a plastic bag for 5 minutes. The mixture is then hand filled into size 5 small transparent gelatin capsules with a filling weight of 100 mg.

A formulation of the coating solution is prepared in a separate container. The composition of the coating suspension is given in the table below.

| Ingredient | Amount (grams) |
|---|---|
| Eudragit E PO | 10.00 |
| Sodium Lauryl Sulfate | 1.05 |
| Magnesium Lauryl Sulfate | 0.55 |
| Polyethylene Glycol 6000 | 2.00 |

To prepare the coating solution, sodium lauryl sulfate is first dissolved in water using a propeller. Eudragit E PO is slowly suspended in this mixture and the stirring continued for another 10 minutes. Then magnesium lauryl sulfate and polyethylene glycol 6000 are added. The suspension is stirred for another 5 hours approximately until a colloidal solution is formed.

The capsules are loaded into a pan coater. The capsules are coated by spraying the coating solution on the capsules sample while the pan is rotated. The air is used and adjusted to keep the sample temperature between 25-35° C. during the coating process.

Capsules are taken periodically and weighed to determine the weight gain. After the desired weight gain of 5% is achieved, the coated capsules are allowed to dry at 35° C. for 2 hours. The pan coater is then turned off and coated capsules are collected for taste testing.

Taste Test: A sample of 10 coated capsules containing approximately 1 gram of the laxative with 5% coating is added to 10 mL of cold water in a cup. After 10 minutes, the capsules are still intact and the water with dispersed capsules has no salty or bitter taste.

### Example 3

A sample of laxative ingredients containing polyethylene glycol 3350 (420 grams), sodium chloride (6 grams), and sodium bicarbonate (11 grams), and potassium chloride (2 grams) is mixed in a plastic bag for 5 minutes. A 50 gram of the mixture was taken and placed in separate glass container. Water in the amount of 5 grams was added into the glass container. The mixture is then heated to about 75° C. until a clear solution is formed. The clear solution is then allowed to cool down to room temperature to form a soft paste. The paste is extruded at room temperature and cut into beads of approximately 1 mm in diameter and 2 mm in length. The extruded granules are placed on an open pan and allowed to dry at room temperature for overnight.

In a separate container, the coating solution is prepared by adding 5 grams of Eudragit E PO and 50 grams of isopropyl ethanol and stirring the sample until a clear solution is formed.

The extruded beads are loaded into a pan coater for coating by spraying the coating solution on the beads while the pan is rotated. Warm air is used during the coating process. The beads are kept between 25-35° C. After all the spray solution is sprayed, the coated beads are allowed to be further dry in an open pan at room temperature for overnight.

Taste Test: A 1 gram sample of the coated capsules is added to 10 mL of cold water in a cup. After 20 minutes, the beads are still intact and the water with dispersed beads has no salty or bitter taste.

### REFERENCES CITED

1, NuLYTELY® with Flavor Packs PEG-3350, Sodium Chloride, Sodium Bicarbonate and Potassium Chloride for Oral Solution, Braintree Laboratories Inc., Prescribing Information, March 2008.
2, CoLYTELY® PEG-3350 and Electrolyte for Oral Solution, Braintree Laboratories Inc., Full Prescribing Information, November 2001.
3. MovipPrep® (PEG-3350, Sodium Sulfate, Sodium Chloride, Potassium Chloride, Sodium Ascorbate and Ascorbic Acid for Oral Solution, Salix Pharmaceuticals, Inc., Prescribing Information, 2006.
4, Barras; Norman, Cox; Ian David, U.S. Pat. No. 7,169,381, Jul. 30, 2007
5. Cleveland; Mark vB. U.S. Pat. No. 6,946,149, Sep. 20, 2005.

What is claimed is:

1. A composition of taste masking solid formulation of laxative comprising at least one ingredient that has a pH-dependent or temperature-dependent solubility in aqueous media.

2. A composition of taste masking solid formulation of claim 1, comprising at least one ingredient that is water insoluble in neutral pH conditions but soluble at acidic pH conditions.

3. A composition of taste masking solid formulation of claim 1, comprising at least one ingredient that has an increased aqueous solubility with reduced pH.

4. A composition of taste masking solid formulation of claim 1, comprising at least one ingredient that has one or more cationic functional groups which increase its aqueous solubility with reduced pH.

5. A composition of taste masking solid formulation of claim 1, comprising of at least one ingredient that is a methacrylic acid copolymer.

6. A composition of taste masking solid formulation of claim 1, comprising of poly(butyl methacrylate, (2-dimethyl aminoethyl)methacrylate, methyl methacrylate).

7. A composition of taste masking solid formulation of claim 1, comprising at least one ingredient that is water insoluble at or below room temperature but soluble in the warm gastric fluid.

8. A composition of taste masking solid formulation of claim 1, comprising at least one ingredient that is gelatin.

9. A composition of taste masking solid formulation of claim 1, comprising at least one ingredient that has a pH-dependent aqueous solubility and one or more other pharmaceutically acceptable excipients that are fully soluble or dispersible in the acidic gastric fluid.

10. A composition of taste masking solid formulation of claim 1, comprising at least one ingredient that has a pH-dependent or temperature-dependent solubility in the coating layer or encapsulation membrane.

11. A composition of taste masking solid formulation of claim 1, comprising at least one ingredient that has an increased aqueous solubility with reduced pH in the coating layer or encapsulation membrane.

12. A composition of taste masking solid formulation of claim 1, comprising at least one ingredient that has one or more cationic functional groups which increase its aqueous solubility with reduced pH in the coating layer or encapsulation membrane.

13. A composition of taste masking solid formulation of claim 1, comprising at least one ingredient that is a methacrylic acid copolymer in the coating layer or encapsulation membrane.

14. A composition of taste masking solid formulation of claim 1, comprising at least poly(butyl methacrylate, (2-dimethyl aminoethyl)methacrylate, methyl methacrylate) in the coating layer or encapsulation membrane.

15. A composition of taste masking solid formulation of claim 1, comprising at least a temperature-dependent solubility in the coating layer or encapsulation membrane.

16. A composition of taste masking solid formulation of claim 1, comprising at least one ingredient that is gelatin in the coating layer or encapsulation membrane.

17. A composition of taste masking solid formulation of claim 1, comprising at least one ingredient that has a pH-dependent or temperature-dependent aqueous solubility and one or more other pharmaceutically acceptable excipients that are fully soluble or dispersible in the warm acidic gastric fluid in the coating layer or encapsulation membrane.

18. The composition of formulation of claim 1, which comprises of 0.1% to 99.0%, preferably 0.5% to 50%, and more preferably 1% to about 20% of the ingredient that has a pH-dependent or temperature-dependent solubility in aqueous media by weight, for taste masking purposes.

19. A composition of taste masking solid formulation of laxative of claim 1, wherein the particles are of the laxative are of any physical shape including but not limited to: sphere, cylinder, cube, rod, plate, capsule-shaped, tablet-shaped, irregular shaped, with a volume average particle size of 1.0 micron to 10 mm in diameter, preferably, 10 microns to 6.0 mm, and more preferably 100 microns to 5.0 mm.

20. A composition of coated capsule formulation of laxative compromising at least one ingredient that has a pH-dependent or temperature-dependent solubility in aqueous media.

21. A composition of taste masking solid formulation of laxative of claim 20, wherein the laxative ingredients are filled into separate capsules for coating.

22. A method for preparing a solid taste masking formulation of laxative containing polyethylene glycol 3350 (PEG 3350) with or without other laxative ingredients comprising the steps of:
(a) heating the PEG 3350 and other laxative ingredients to allow the PEG 3350 to melt;
(b) allowing the molten material to cool until it forms a paste;
(c) extruding the paste into appropriate shaped particles before it becomes hard solid;
(d) coating the extruded particles by a spray coating process with the taste masking coating suspension or solution.

23. A method according to claims 22, wherein water is added to the PEG 3350 and other ingredients mixture during at least one of the first three steps, step (a), (b), or (c).
